(19) United States
(12) Patent Application Publication
(10) Pub. No.: US 2024/0013900 A1
(43) Pub. Date: Jan. 11, 2024

(54) ACTIVE PATIENT-SPECIFIC MONITORING SYSTEM

(71) Applicant: Becton, Dickinson and Company, Franklin Lakes, NJ (US)

(72) Inventors: Vikas Gupta, Naperville, IL (US); Kalvin Yu, Los Angeles, CA (US)

(21) Appl. No.: 18/022,477

(22) PCT Filed: Aug. 20, 2021
(86) PCT No.: PCT/US21/46871

§ 371 Date: Feb. 21, 2023

Related U.S. Application Data
(60) Provisional application No. 63/068,921, filed on Aug. 21, 2020.

(51) Int. Cl. G16H 40/40; G16H 10/60; G16H 20/13; G16H 40/20

(52) U.S. Cl. CPC G16H 40/40; G16H 10/60; G16H 20/13; G16H 40/20

(57) ABSTRACT

A method of configuring a medical device includes receiving first information characterizing a health condition of a patient. The method includes retrieving, from a model data storage device, a generic model, which receives, as an input, patient information, and provides, as an output, a generic configuration for a medical device. The method also includes generating based at least in part on the generic model and the health condition of the patient, a patient-specific model. The method also includes providing the first information to the patient-specific model to generate a patient-specific configuration for the medical device. The method also includes presenting a configuration recommendation. The configuration recommendation includes the generated patient-specific configuration. Related methods and articles of manufacture, including apparatuses and computer program products, are also disclosed.

### CROSS-REFERENCE TO RELATED APPLICATIONS

This application claims priority to and is a non-provisional application of U.S. Provisional Application Ser. No. 63/068,921, entitled “ACTIVE PATIENT SPECIFIC MONITORING SYSTEM,” filed on Aug. 21, 2020, the entirety of which is hereby incorporated by reference.

### TECHNICAL FIELD

The subject matter described herein relates generally to patient monitoring and medication delivery systems and more specifically to a patient monitoring system for configuring a medical device and recommending dosages.

### BACKGROUND

Therapy may be administered to patients by delivering a medication to the patient using one or more medical devices, such as infusion pump systems, or by manually providing a dosage of the medication to the patient. Pharmacokinetic and pharmacodynamics (Pk/Pd) predictive models may be used in patient monitoring systems to calculate medical device configurations and to calculate appropriate medication dosages that are used to treat patients. These predictive models may rely on general clinical and infusion data to provide the medical device configuration and/or a recommended dosage. However, these systems may not account for patient conditions that change over time and over the course of an administered therapy. This may lead to preparing and/or delivering an incorrect dose of the medication to the patient or administering a dose that causes unintended consequences as a result (e.g., an adverse drug event (ADE), poor clinical response/outcome, etc.).

### SUMMARY

Systems, methods, and articles of manufacture, including computer program products, are provided for configuring a medical device and recommending dosages based at least in part on a health condition of a patient. For example, the system may provide more accurate medication preparation and delivery configurations and/or dosages for a patient to more effectively, efficiently, and quickly treat the patient.

According to some aspects, a method includes receiving first information characterizing a health condition of a patient. The method may also include retrieving, from a model data storage device, a generic model. The generic model may receive, as an input, medication information, and the generic model may provide, as an output, a generic configuration for the medical device. The method may include generating, based at least in part on the generic model and the health condition of the patient, a patient-specific model. The method may include providing the first information to the patient-specific model to generate a patient-specific configuration for the medical device. The method may also include presenting a configuration recommendation. The configuration recommendation may include the generated patient-specific configuration.

In some aspects, the method includes identifying the medical device associated with the patient. The method may also include transmitting, to the medical device associated with the patient, a control message including the patient-specific configuration.

In some aspects, the patient-specific configuration is different from the generic configuration.

In some aspects, the method includes requesting confirmation that the generated patient-specific configuration should be transmitted to the medical device.

In some aspects, the method includes receiving, in response to the requested confirmation, the confirmation.

In some aspects, the method includes receiving, in response to the requested confirmation, an adjustment of the patient-specific configuration.

In some aspects, the method includes generating the patient-specific configuration for the medical device.

In some aspects, the method includes receiving second information characterizing a second health condition of the patient at a second time that is after a first time of the first health condition. The method may also include generating, based at least in part on the patient-specific model and the second information, an updated patient-specific model. The method may also include providing the second information to the updated patient-specific model to generate an updated patient-specific configuration for the medical device.

In some aspects, the method includes requesting confirmation that the updated patient-specific configuration should be provided to the medical device.

In some aspects, the method includes generating an alert indicating that an updated patient-specific configuration has been generated.

In some aspects, the method includes determining that the second health condition is different from the first health condition.

In some aspects, the method includes generating an alert indicating that the patient-specific configuration should be updated.

In some aspects, the method includes causing a change in operation of the medical device.

In some aspects, the method includes causing the medical device to stop operation according to the patient-specific configuration.

In some aspects, the method includes causing the medical device to operate according to the updated patient-specific configuration.

In some aspects, the first information includes patient data. The patient data may represent one or more of a genetic test result, a genomic test result, a change in organ function of the patient, a severity of illness of the patient, a type of health condition of the patient, an allergy of the patient, a medication prescribed for the patient, a liver function test result, a kidney function test result, a molecular test result, a drug level test result, an abnormal chemistry/electrolyte lab value or an abnormal hematology lab value including an abnormal blood cell panel.

In some aspects, the patient-specific model is further generated based at least in part on one or more patient demographics. The one or more patient demographics may include a patient age, a patient sex, a patient race, a patient ethnicity, a patient height, a patient weight, and a patient gender. In some aspects, the patient-specific model is further generated based at least in part on whether the health condition of the patient is improving or worsening. In some aspects, the patient-specific model is further generated based at least in part on medical device data. The medical device data may include one or more medication-specific or diagnostic-specific information. In some aspects, the generic model includes one or more of a pharmacokinetic model, a pharmacodynamics model, and a Bayesian-based model.

In some aspects, the method includes causing, based on the patient-specific configuration information, administration of a medication to the patient,

According to some aspects, a method may include receiving first information characterizing a health condition of a patient. The method may also include retrieving, from a model data storage device, a generic model. The generic model may receive, as an input, medication information for a medication, and provide, as an output, an initial dosage for the medication to be delivered to the patient. The method may further include generating a patient-specific model based at least in part on the generic model and the health condition of the patient. The method may also include providing the first information to the patient-specific model to generate a patient-specific dosage for the medication. The method may also include presenting a dosage recommendation for the medication. The dosage recommendation may include the patient-specific dosage.

In some aspects, the method includes determining that the patient-specific dosage is different from the initial dosage.

In some aspects, the method includes generating, based on the determination, an alert indicating that the patient-specific dosage is different from the initial dosage.

In some aspects, the method includes requesting, after determining that the patient-specific dosage is different from the initial dosage, confirmation of the patient-specific dosage.

In some aspects, the method includes transmitting, based on the confirmation of the patient-specific dosage, the patient-specific dosage to a medical device associated with the patient.

In some aspects, the method includes transmitting, based on the confirmation of the patient-specific dosage, the patient-specific dosage to a dispensing cabinet. The dispensing cabinet may allow the patient-specific dosage to be dispensed.

In some aspects, the method includes transmitting, based on the confirmation of the patient-specific dosage, the patient-specific dosage to a pharmacy information system.

In some aspects, the patient-specific dosage includes one or more of a medication type, an amount of the medication, a time frequency for delivering the medication, and an amount of time for delivering the medication.

According to some aspects, a method includes receiving information characterizing a health condition of one or more patients at a medical facility. The method may also include generating, based on the information, a metric for the medical facility. The method may further include determining whether the metric corresponds to a safety threshold. The method may also include scheduling, based on the determination, a next review for the facility.

In some aspects, the method also include determining that the metric does not correspond to the safety threshold. The method may also include identifying a facility configuration for updating. The method may also include initiating an update of the facility configuration.

In some aspects, the method also includes determining that the metric corresponds to the safety threshold.

Embodiments of the current subject matter can include methods consistent with the descriptions provided herein as well as articles that comprise a tangibly embodied machine-readable medium operable to cause one or more machines (e.g., computers, etc.) to result in operations implementing—or signaling the need to implement—one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more embodiments of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed embodiments. In the drawings,

FIG. 1A depicts a system diagram illustrating a patient monitoring system, in accordance with some example embodiments;

FIG. 1B depicts another system diagram illustrating a patient monitoring system, in accordance with some example embodiments;

FIG. 2 schematically depicts an example display, in accordance with some example embodiments;

FIG. 3 depicts a flowchart illustrating a process for configuring a medical device, in accordance with some example embodiments;

FIG. 4 depicts a flowchart illustrating a process for providing a dosage for a medication, in accordance with some example embodiments;

FIG. 5 depicts a flowchart illustrating a process for generating a metric for a facility;

FIG. 6 depicts a block diagram illustrating a computing system, in accordance with some example embodiments;

FIG. 7A depicts a front view of a patient care system, in accordance with some example embodiments;

FIG. 7B depicts an enlarged view of a portion of a patient care system, in accordance with some example embodiments;

FIG. 7C depicts a perspective view of a pump, in accordance with some example embodiments; and

FIG. 8 depicts messages that may be exchanged to configure a medical device.

When practical, similar reference numbers denote similar structures, features, or elements.

### DETAILED DESCRIPTION

Therapy may be administered to patients by delivering a medication to the patient using one or more medical devices, such as infusion pump systems, or by manually providing a dosage of the medication to the patient. Pharmacokinetic and pharmacodynamics (Pk/Pd) predictive models (e.g., Eleveld propofol, remifentanil TCI models, Bayesian-based models, and/or the like) may be used in patient monitoring systems. For example, the Pk/Pd predictive models may be loaded onto and stored on a medical device, may be used to generate medical device configurations to be transmitted to a medical device, and/or may calculate or suggest appropriate medication dosages that may be optimal in the treatment of patients. In order to facilitate an optimal treatment, a clinician may select a desired Pk/Pd model, and based on the selected model, a medical device configuration may be transmitted to the medical device to begin delivering the medication to the patient and/or a medication dosage may be ordered to be prepared or accessed. Thus, these predictive models directly impact a prescribing healthcare professionals' choice of dose of the medication that is delivered to the patient.

These predictive models may rely solely on general clinical and infusion data or stored electronic medical records to provide the medical device configuration and/or appropriate medication dosage. However, systems incorporating these models may not account for patient conditions that change over the course of an administered therapy. Also, the data and/or stored electronic medical records, upon which these predictive models are based, may be inaccurate. This may lead to delivering an incorrect dose of the medication to the patient, ineffectively treating the patient, and in some cases contributing to a worsening condition of the patient.

The patient monitoring system, consistent with embodiments of the current subject matter may address one or more of these issues, by, for example, generating one or more patient-specific models based at least in part on a patient condition and/or a change in the patient condition. Generation of the patient-specific models may additionally and/or alternatively be based at least in part on device-specific data, such as medication-specific and diagnostic-specific information collected from a medical device. Thus, the patient monitoring system described herein may provide dynamic guardrails that increase precision and patient safety by tailoring medical device configurations and recommended dosages to treat specific patient health conditions that may change over the course of the administered therapy. The patient monitoring system described herein thus helps to minimize the risk of an adverse event, such as toxicity, adverse interactions between drugs, end organ damage and/or other measurable poor outcomes caused by under-dosing or over-dosing. Additionally and/or alternatively, the patient monitoring system described herein may improve real-time patient management and/or provide metrics related to performance of therapy programs. For example, the patient monitoring system described herein may provide patient specific dosing strategies and related population based metrics to allow clinicians to optimize local guidelines and/or patient specific opportunities to intervene during administration of a therapy.

The patient monitoring features may additionally or alternatively be applicable to preparation of medications. For example, absent patient specific information, a standard dose of a medication may be prepared. The standard dose may require specific (and sometimes) limited resources such as expensive or hazardous ingredients, instrument time to prepare the dose, etc. In some instances, a patient-specific preparation may not require as many resources and thereby conserve through consideration of patient specific aspects, such as those described, during the preparation process.

The patient monitoring features may additionally or alternatively be applicable to diagnostic medical devices and systems. For example, a diagnostic device such as the BD KIESTRA™ system or the BD MAX™ system or the BD VERITOR™ system or the BD PHOENIX™ system may analyze samples taken from a patient. The analysis may be tailored using patient specific aspects, such as those described. The tailoring may include, for example, adjusting detection thresholds, control of the analysis hardware (e.g., time to test, time to result, time to read, detection range or spectrums, prism focusing, etc.), or the like. Information generated by a diagnostic device may be included in the patient specific analysis. In some instances, the inclusion may be “real-time.” As used herein, “real-time” may refer to availability for processing at or near in time to the time the associated item is generated or detected.

The patient monitoring features may additionally or alternatively be applicable to monitoring devices and systems. For example, a monitoring device such as a digital catheter may analyze fluid output from a patient. The analysis may be tailored using patient specific aspects, such as those described. The tailoring may include, for example, adjusting detection thresholds (e.g., over production, under production), control of the monitoring hardware (e.g., detection range or spectrums, alert thresholds, frequency of monitoring, quantity of measurements to retain, etc.), or the like. In some implementations, the features may be implemented in a clinical decision support module that controls one or more of the devices described based at least in part on information received from a monitoring device. Information generated by a monitoring device may be included in the patient specific analysis. In some instances, the inclusion may be “real-time.”

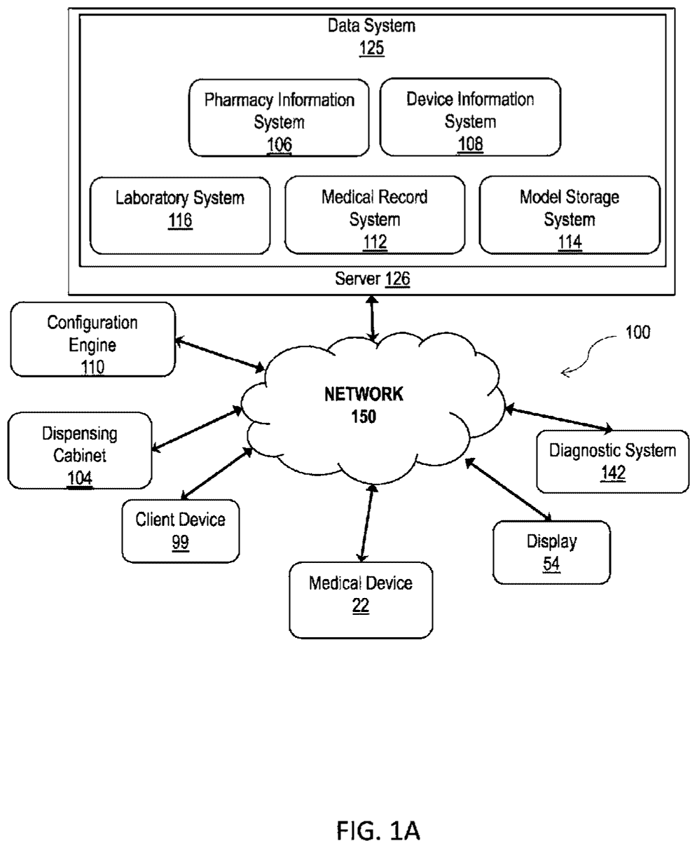

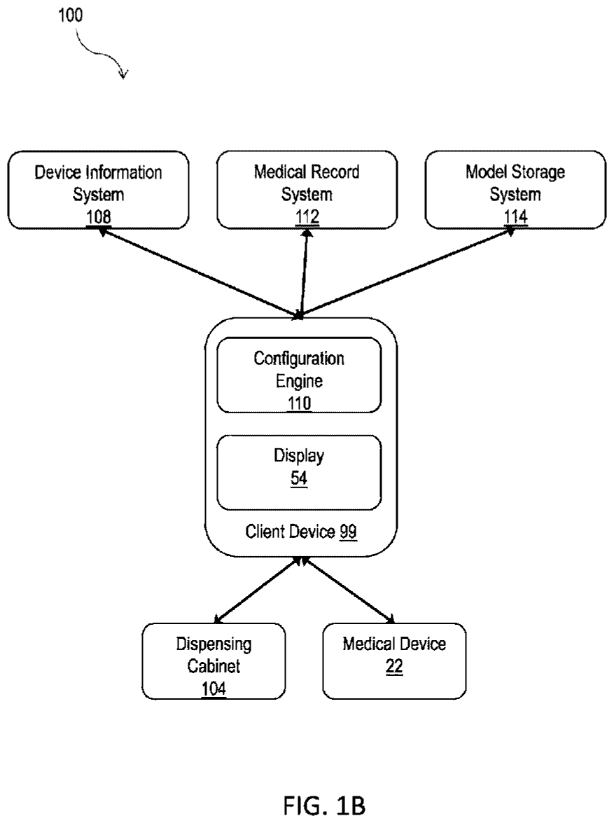

FIG. 1A depicts a system diagram illustrating a patient monitoring system 100, in accordance with some example embodiments. Referring to FIG. 1A, the patient monitoring system 100 may include a configuration engine 110, a medical device 22, a display 54, a client device 99, a dispensing cabinet 104, a diagnostic system 142, and/or a data system 125. In some example embodiments, the configuration engine 110, the display 54, the client device 99, the diagnostic system 142, and/or the data system 125 may form a portion of the medical device 22 and/or may be positioned within a housing of the medical device 22. Additionally and/or alternatively, the configuration engine 110, the display 54, the client device 99, the diagnostic system 142, and/or the data system 125 may form a portion of the dispensing cabinet 104 and/or may be positioned within a housing of the dispensing cabinet 104.

Referring to FIG. 1A, the configuration engine 110, the medical device 22, the display 54, the client device 99, the dispensing cabinet 104, the diagnostic system 142, and/or the data system 125 may be communicatively coupled via a network 150 and/or via a direct device-device connection as described herein. The network 150 may be a wired and/or wireless network including, for example, a public land mobile network (PLMN), a local area network (LAN), a virtual local area network (VLAN), a wide area network (WAN), the Internet, a short range radio connection, for example Bluetooth, a peer-to-peer mesh network, and/or the like.

The display 54 may form a part of the medical device 22 or may be separately coupled as part of the client device 99. The display 54 may also include a user interface. The user interface may form a part of a display screen of the display 54 that presents information to the user (e.g., a clinician, a patient, or caregiver for the patient) and/or the user interface may be separate from the display screen. For example, the user interface may be one or more buttons, or portions of the display screen that is configured to receive an entry from the user.

The client device 99 may be a mobile device such as, for example, a smartphone, a tablet computer, a wearable apparatus, and/or the like. However, it should be appreciated that the client device 99 may be any processor-based device including, for example, a desktop computer, a laptop or mobile computer, a workstation, and/or the like. For example, via the client device 99, the user may be able to configure certain parameters of the medical device 22, such as an air in line threshold, a rate limit, an alarm limit, and the like. Additionally, in some examples, via the client device 99, the user may configure various medical device configurations, medication dosages or protocols, and/or the like.

The data system 125 may include one or more databases, providing physical data storage within a dedicated facility and/or being locally stored on the medical device 22, the dispensing cabinet 104, the diagnostic system 142, and/or the client device 99. Additionally and/or alternatively, the data system 125 may include cloud-based systems providing remote storage of data in, for example, a multi-tenant computing environment and/or the like. The data system 125 may also include non-transitory computer readable media.

The data system 125 may include a medical record system 112, a pharmacy information system 106, a device information system 108, a model storage system 114, and/or a laboratory system 116. The medical record system 112 may include an inventory or dispensing system, a patient care system, an administrative system, an electronic medical record system, and/or the like, which store a plurality of electronic medical records, each of which include the patient's medical history, one or more patient parameters (including laboratory results), one or more medication delivery protocols (including one or more medication dosages), and/or the like. The device information system 108 may store data such as medication-specific and diagnostic-specific information, such as device and/or medication preparation, dispensing, diagnostic assay results, and infusion data, collected from the medical device 22. The pharmacy information system 106 may include a medication library, which stores a list of available medications, and/or medication names associated with recommended dosages and dose limits that have been established or adopted by a healthcare facility. In some embodiments, the pharmacy information system 106 is accessible to a clinician at the healthcare facility and/or to a pharmacist at another facility, such as a pharmacy. The laboratory system 116 may store laboratory information such as one or more laboratory results, one or more laboratory orders, and/or the like.

The model storage system 114 may include a model data storage device and may store one or more predictive models, such as the Pk/Pd predictive models and/or Bayesian-based models or other models. The stored predictive models may include one or more generic models, which have been generated based on medication types and/or certain generic patient information. Additionally and/or alternatively, the stored predictive models may include one or more patient-specific models generated by the patient monitoring system 100, consistent with embodiments of the current subject matter.

The data system 125 may include and/or be coupled to a server 126, which may be a server coupled to a network, a cloud server, and/or the like. The medical device 22, the dispensing cabinet 104, and/or the client device 99 may wirelessly communicate with the server 126. The server 126, which may include a cloud-based server, may provide and/or receive data and/or instructions from the data system 125 to the medical device 22, the dispensing cabinet 104, and/or the client device 99, to implement one or more features of the patient monitoring system 100 consistent with embodiments of the current subject matter. Additionally and/or alternatively, the server 126 may receive data (e.g., patient information, information characterizing a health condition of a patient, medication information, trend information (e.g., improving measurements over time), and/or the like) from the medical device 22, the dispensing cabinet 104, and/or the client device 99.

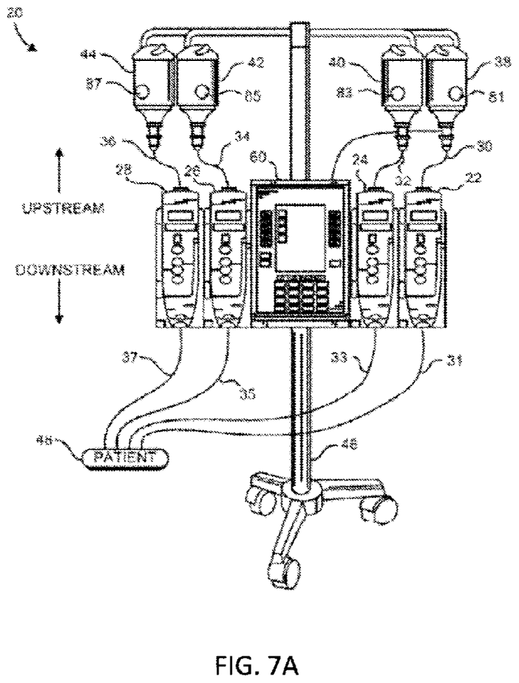

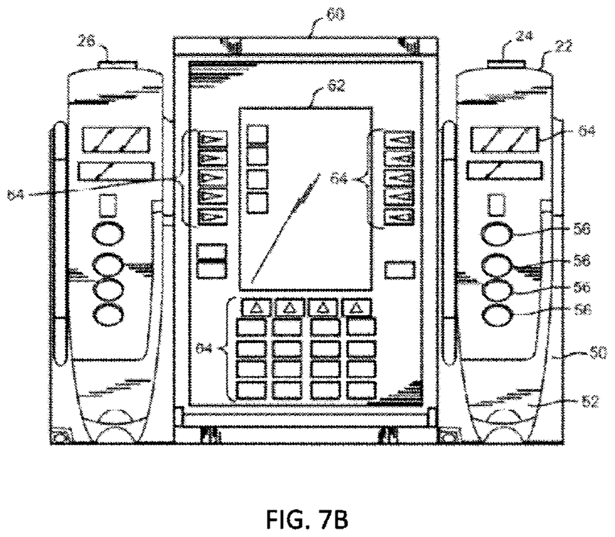

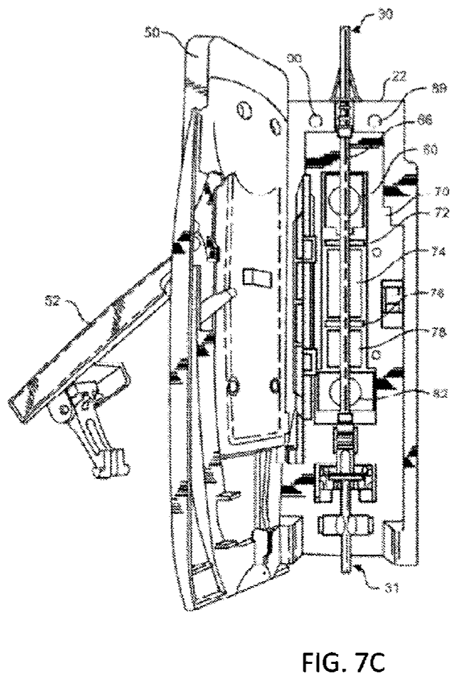

The medical device 22 may be a pump or other infusion device, such as a target-controlled infusion pump, a syringe pump, an anesthesia delivery pump, and/or a patient-controlled analgesic (PCA) pump configured to deliver a medication to a patient. One or more Pk/Pd or other predictive models may be stored on the medical device 22. It should be appreciated that the medical device 22 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like. Alternatively, the medical device 22 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like. Moreover, the medical device 22 may be part of a patient care system, such as the patient care system shown in FIGS. 7A-7C, which includes one or more additional pumps.

The medical device 22 may determine the optimal dose of the medication to deliver to the patient based on the values of one or more patient parameters, including the patient's age, height, weight, gender, laboratory results, whether the patient has ingested opiates, the patient's BMI, and the like, and/or monitoring device information, such as vital signs, monitored patient parameters, and/or the like. In some embodiments, the medical device 22 may display the one or more patient parameters and/or the monitoring device information on the display 54. Before determining the optimal dose of the medication and/or delivering the medication to the patient, such as during initialization of the medical device 22, the medical device 22 may display, via the display 54, a request to the user for the user to enter and/or confirm a value of each of the one or more patient parameters and/or the monitoring device information.

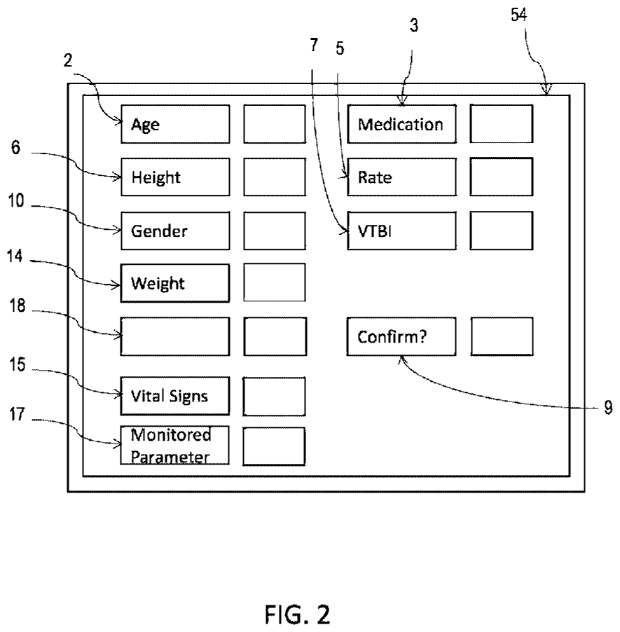

FIG. 2 schematically illustrates an example of the display 54, which can form a part of or be separately coupled to the client device 99, the medical device 22, and/or the dispensing cabinet 104. As shown in FIG. 2, one or more patient parameters may be displayed on the display 54. The one or more patient parameters may include a first patient parameter 2, a second patient parameter 6, a third patient parameter 10, and a fourth patient parameter 14. In some embodiments, the one or more patient parameters also includes a fifth patient parameter 18 and/or more patient parameters. The first patient parameter 2 may correspond to the patient's age, the second patient parameter 6 may refer to the patient's height, the third patient parameter 10 may refer to the patient's gender, the fourth patient parameter 14 may refer to the patient's weight, and the fifth patient parameter 18 may refer to the patient's body mass index (BMI), for example. The patient parameters may also be arranged in other orders.

Again referring to FIG. 2, the display 54 may additionally and/or alternatively display one or more dosage parameters of the therapy to be administered to the patient. The one or more dosage parameters of the therapy may include a type of medication 3, a delivery rate 5, a Volume to Be Infused (VTBI) 7, and/or the like. Additionally and/or alternatively, the display 54 may display monitoring device information. The monitoring device information may include one or more vital signs 15 of the patient, one or more monitored patient parameters 17, a status of the monitoring device, and/or the like.

In some embodiments, the display 54 may also display a request 9 to confirm the one or more patient parameters, the one or more dosage parameters of the therapy, the monitoring device information, and/or the like. For example, the values of the dosage parameters of the therapy may be received via the display 54 after a user interaction with the display and/or may be calculated and presented as part of a selected predictive model. Similarly, the values of the monitoring device information may be received via the display 54 after a user interaction with the display and/or may be recorded from a monitoring device. The display 54 may receive the user's confirmation of the displayed value for each of the values of the dosage parameters 3, 5, 7, and/or the display 54 may receive the user's entry or selection of the value for each of the dosage parameters 3, 5, 7. The display 54 may additionally or alternatively receive the user's confirmation of the displayed value for each of the values of the monitoring information 15, 17, and/or the display 54 may receive the user's entry or selection of the value for each of the the monitoring information 15, 17.

The display 54 (e.g., a dynamic display) also improves the manner in which the client device 99, the medical device 22, and/or the dispensing cabinet 104 displays information and interacts with the user. By dynamically generating values based on an initial input, the client device 99, the medical device 22, and/or the dispensing cabinet 104 may reduce the need to render additional complex data entry elements to complete programing. For example, the graphical user interface presented by the display may include graphical elements to increment or decrement a value of the displayed parameters rather than presenting a full keypad for data entry. The client device 99, the medical device 22, and/or the dispensing cabinet 104 may more efficiently process and validate these input signals, which may be more than entries from freeform text or numeric data entry fields. The use of smaller entry elements also conserves display area on the client device 99, the medical device 22, and/or the dispensing cabinet 104. This permits presentation of more programming parameters at the time of data entry thereby further reducing the likelihood of a programming error.

FIG. 1B schematically illustrates the patient monitoring system 100 consistent with embodiments of the current subject matter. As shown in FIG. 1B, the client device 99, which may form a part of and/or be separate from and communicatively coupled to the dispensing cabinet 104 and/or the medical device 22, may include the configuration engine 110 and/or the display 54. The client device 99 may be communicatively coupled with the device information system 108, the medical record system 112, and/or the model storage system 114.

In some embodiments, the patient monitoring system 100 may generate a patient-specific configuration and configure the medical device 22 with the patient-specific configuration to more effectively, efficiently, and quickly deliver a medication to a patient and to treat the patient. As noted above, the patient monitoring system 100 may dynamically incorporate a patient's health condition and/or a change in the patient's health condition, clinical data, device data, and/or the like, to ensure that the appropriate dosage is being delivered to the patient.

Referring to FIG. 1B, the configuration engine 110 may receive a first set of information characterizing a health condition of the patient. The configuration engine 110 may receive the first set of information upon initialization of a medical device, upon creation of an order by a clinician, during administration of a therapy to a patient, after performing one or more tests on the patient, and/or the like. The first information may be retrieved from the medical record system 112 and/or the device information system 108. In some embodiments, the first information is stored (e.g., temporarily) on the client device 99. The first information may be dynamically updated as the health condition of the patient changes, such when test results, device data, and/or other patient data are received and/or updated at the medical record system 112 and/or the device information system 108. The first information may include patient data, such as a genetic test result, a genomic test result, a change in organ function of the patient, a severity of illness of the patient, an initial diagnosis or type of the health condition of the patient, an allergy of the patient, a medication prescribed for the patient, a liver function test result, a kidney function test result, a blood cell panel and/or the like.

Based at least on the initial health condition of the patient and/or a selected medication, the configuration engine 110 may retrieve, from the model storage system 114, a generic model, such as a Pk/Pd model and/or other model (such as a Bayesian-based model), from the stored predictive models. The model can be singular or multiple in nature. The generic model may receive one or more inputs, such as the initial health condition of the patient and/or the selected medication to administer to the patient, and provide one or more outputs, such as a generic configuration for the medical device and/or an initial dosage recommendation for the medication to be delivered to the patient. In some embodiments, the client device 99 may receive, via the display 54, a selection of the generic model from the stored predictive models, and the configuration engine 110 may retrieve the selected generic model from the model storage system 114.

In some embodiments, the configuration engine 110 may generate a patient-specific model, which incorporates the health condition of the patient, a change in the health condition of the patient, medical device data, a change in the medical device data, and/or the like, to improve administration of the therapy to the patient. For example, the configuration engine 110 may generate the patient-specific model based at least in part on the generic model and the health condition of the patient (e.g., a dynamically changing health condition of the patient), one or more patient demographics and/or parameters, such as the patient's age, sex, race, ethnicity, height, weight, laboratory results, and/or gender, whether the health condition of the patient is improving or worsening, medical device data (e.g., dynamically changing medical device data such as medication-specific or diagnostic-specific information), a previous patient-specific model, monitoring device information, such as the patient's vital signs, monitored patient parameters, and/or the like. Once the patient-specific model is generated, the configuration engine 110 may provide the first set of information to the patient-specific model and the patient-specific model may generate a patient-specific configuration for the medical device and/or a recommended dosage for the medication. The patient-specific configuration for the medical device and/or the recommended dosage for the medication may include a medication type, an amount of the medication, a time frequency for delivering the medication, and amount of time for delivering the medication and/or the like. Additionally and/or alternatively, the patient-specific configuration for the medical device and/or the recommended dosage may be different from and more accurate than the generic configuration and/or the initial dosage output by the generic model, as the patient-specific model incorporates at least the health condition of the patient.

In some embodiments, the configuration engine 110 generates an alert indicating that the patient-specific configuration or dosage is different than the generic configuration or initial dosage, that the patient-specific configuration or patient-specific dosage has been generated, and/or that the patient-specific configuration or dosage should be updated because the current configuration or dosage may no longer be appropriate. The alert may include an audio (e.g., a sound, a patterned sound, and/or the like) and/or visual (e.g., a light, a flashing light, a colored light, a patterned light, and/or the like) indicator.

In some embodiments, the configuration engine 110 displays, via the display 54, a configuration recommendation, which includes the generated patient-specific configuration. For example, as shown in FIG. 2, the display 54 may present one or more dosage parameters 3, 5, 7, and request confirmation that the generated patient-specific configuration should be transmitted to the medical device. Via the display 54, the user may confirm the configuration recommendation and/or adjust one or more values of the dosage parameters 3, 5, 7. After receiving the confirmation of the configuration recommendation, the configuration engine 110 may identify the medical device 22 associated with the patient and transmit a control message, including the patient-specific configuration, to the medical device 22.

Additionally and/or alternatively, the configuration engine 110 displays, via the display 54, a dosage recommendation, which includes the generated patient-specific dosage. For example, as shown in FIG. 2, the display 54 may present one or more dosage parameters 3, 5, 7, and request confirmation that the generated patient-specific dosage should be transmitted to the medical device to configure the medical device, the dispensing cabinet to allow access to the dosage of the medication, a pharmacy for preparing the dosage of the medication, and/or the like. Via the display 54, the user may confirm the dosage recommendation and/or adjust one or more values of the dosage parameters 3, 5, 7. After receiving the confirmation of the dosage recommendation, the configuration engine 110 may identify the medical device 22 associated with the patient, the dispensing cabinet 104, and/or the pharmacy, and transmit a control message, including the patient-specific dosage, to the medical device 22, the dispensing cabinet 104, and/or the pharmacy. The transmitted control message may, in some instances, cause the medical device 22 to begin administration of the medication to the patient.

As described herein, the health condition of the patient may be dynamically updated to represent the current health condition of the patient. In some embodiments, the configuration engine 110 may receive a second set of information characterizing a second health condition of the patient at a second time that is after a first time of the initial health condition. The configuration engine 110 may generate, based at least in part on the patient-specific model and the second set of information, an updated patient-specific model.

In some embodiments, the configuration engine 110 may provide the second set of information to the updated patient-specific model to generate an updated patient-specific configuration and/or dosage. The configuration engine 110 may then request confirmation that the updated patient-specific configuration or dosage should be provided to the medical device 22, the dispensing cabinet 104, and/or the pharmacy. Additionally and/or alternatively, the configuration engine 110 may generate an alert indicating that the patient-specific configuration should be updated. Additionally and/or alternatively, the configuration engine 110 may cause a change in operation of the medical device, such as upon detection of a change in the health condition of the patient and/or a change in the patient-specific model and/or the patient-specific configuration and/or dosage. For example, the configuration engine 110 may determine that the health condition of the patient has changed such that the current patient-specific configuration and/or dosage is incorrect. The configuration engine 110 may then generate an alert, cause a change in the dosage of the medication recommended or administered to the patient, and/or cause a change in operation of the medical device, such as causing the medical device to stop operation according to the patient-specific configuration and/or causing the medical device to operate according to an updated patient-specific configuration. Accordingly, the patient monitoring system described herein may more accurately and effectively treat patients.

In some embodiments, the patient monitoring system includes a metrics platform. The metrics platform may provide and/or generate, based on at least the records stored in the medical record system 112, the records stored in the device information system 108, the health condition of the patients, and/or the like, one or more metrics. The one or more metrics may be used to benchmark safety issues at a medical facility. For example, the one or more metrics may indicate whether a medical facility is following internal protocols or best practice guidelines and/or may be used to compare the efficacy of medical facilities in their ability to treat patients having certain health conditions. Thus, the one or more metrics may indicate whether a medical facility should update its protocols and/or models for medication dosing. In some embodiments, the one or more metrics includes an amount of wasted medication, which may indicate how well the medications were prepared, an amount of time taken to prepare a medication for administration, and/or the like. The one or more metrics may additionally and/or alternatively evaluate whether a generated dosage is within a target range, whether the generated dosage is above the target range, and/or whether the generated dosage is below the target range. This may help to better evaluate patient response to certain types of configurations and dosages, which helps to improve the predictive models described herein.

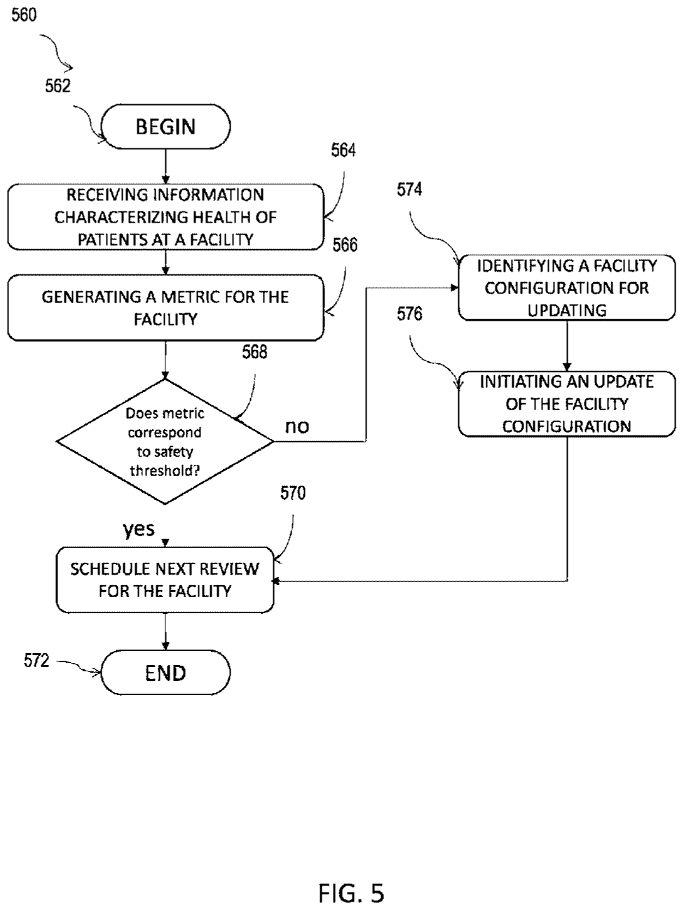

FIG. 5 depicts a flowchart illustrating a process 560 for generating a metric for a medical facility. At 562, the process for generating the metric may begin. For example, the patient monitoring system 100 (including the metrics platform) may be initialized. Initialization may include obtaining or generating threshold values used in the process 560, establishing one or more connections with data sources for inputs to the process 560, identify a model for processing the inputs to the process 560, or the like.

At 564, the patient monitoring system 100, such as via the configuration engine 110, may receive information (e.g., first information) characterizing a health condition of one or more patients at the medical facility. The information may include patient data representing a genetic or molecular test result, a genomic test result, a change in organ function of the patient, a severity of illness of the patient, a type of health condition of the patient, an allergy of the patient, a medication prescribed for the patient, a liver function test result, a kidney function test result, and/or a blood cell panel for each of the patients.

At 566, the configuration engine 110 may, based on the received information, generate a metric for the medical facility. As noted above, the metric may include compliance of the medical facility with internal protocols or best practice guidelines. Additionally and/or alternatively, the metric may include a comparison of patient metrics at the medical facility to a standard and/or a generic patient metric. Thus, the metric may be used to compare the efficacy of medical facilities in their ability to treat patients having certain health conditions.

At 568, the configuration engine 110 determines whether the metric corresponds to (e.g., is less than, greater than, and/or equal to) a safety threshold. The safety threshold may include a compliance score indicating the medical facility's compliance with its protocols and/or best practices guidelines, a tolerance for deviation from the standard and/or generic patient metric (e.g., 1% deviation, 5% deviation, 10% deviation, 15% deviation, etc.), and/or the like. The threshold may be a static value provided as a configuration to the system. In some implementations, the threshold may be dynamically generated to tailor the safety parameter to specific characteristics of the facility (e.g., number of beds, number of clinicians, average patient stay duration, age of facility, etc.) and/or patient(s) under analysis (e.g., typical condition, medication dispensed, demographics, etc.).

If the configuration engine 110 determines that the metric does not correspond to the safety threshold, at 574, the configuration engine 110 may identify a facility configuration for updating. The facility configuration to be updated may include a procedure, an internal protocol, a best practices guidelines, a generic model, a patient-specific model, a device configuration (e.g., settings, default parameters, etc.), and/or the like. As an example, the configuration engine 110 may update one or more generic models upon which the generated patient-specific models are based.

At 576, the configuration engine 110 may then initiate an update of the facility configuration. For example, initiating the update of the facility configuration may include transmitting one or more control messages to an associated device (e.g., one or more client devices 99, one or more dispensing cabinets 104, one or more medical devices 22, and/or the like). The control messages may cause the medical device to administer the medication to the patient according to an updated facility configuration, may change the generic models, may change the patient-specific models, may cause one or more device configurations (e.g., settings, default parameters, etc.) to change, may cause the medical device to stop administration of the medication to one or more of the patients in the medical facility, may generate an alert at the associated device, and/or the like.

If the configuration engine 110 determines that the metric does correspond to the safety threshold at 568, identifies a facility configuration for updating at 574, and/or initiates an update of the facility configuration, the configuration engine 110 may schedule a next review for the medical facility at 570. The configuration engine 110 may schedule the next review based on an amount the protocols of the medical facility. For example, if internal protocols and/or best practices guidelines of a medical facility are significantly updated and/or are updated by an amount that is equal to or exceeds a threshold amount, the next review may be scheduled more often and/or sooner to increase the amount of monitoring of the medical facility to ensure the medical facility is safely operating. Additionally and/or alternatively, the configuration engine 110 may schedule the next review based on a degree of deviation from the safety threshold. If the configuration engine 110 determines that the metric is within a threshold amount and/or exceeds the safety threshold by a threshold amount (e.g., within 1%, 5%, 10%, 15%, etc.), the next review may be scheduled more often and/or sooner to increase the amount of monitoring of the medical facility to ensure the medical facility is safely operating. If the configuration engine 110 determines that the metric is not within a threshold amount and/or is less than the safety threshold by a threshold amount (e.g., within 1%, 5%, 10%, 15%, etc.), the next review may be scheduled less often, as the medical facility is operating safely. At 572, the metrics platform and/or the patient monitoring system ends the process 560.

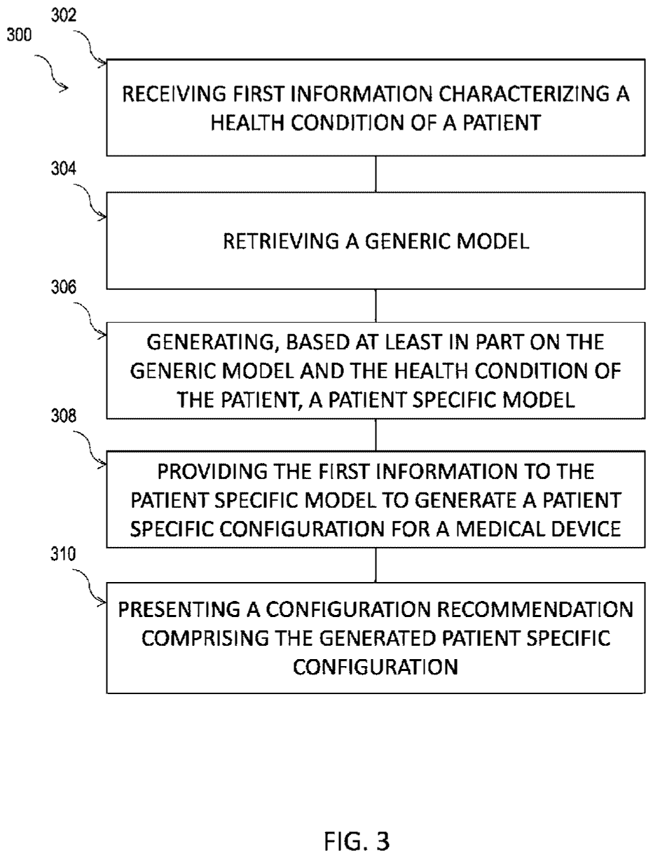

Referring back to FIG. 3, FIG. 3 depicts a flowchart illustrating a process 300 for configuring a medical device, consistent with embodiments of the current subject matter. Referring to FIG. 3, the process 300 may be performed by the patient monitoring system 100, such as by the configuration engine 110.

In some embodiments, the patient monitoring system (e.g., the patient monitoring system 100) is initialized. At 302, the patient monitoring system, such as the configuration engine, may receive first information characterizing a health condition of a patient. The first information may include patient data representing a genetic or molecular test result, a genomic test result, a change in organ function of the patient, a severity of illness of the patient, a type of health condition of the patient, an allergy of the patient, a medication prescribed for the patient, a liver function test result, a kidney function test result, and/or a blood cell panel.

At 304, a generic model (e.g., a predictive model including a Pk/Pd model and/or a Bayesian-based predictive model and/or other models) may be retrieved by the configuration engine from a model data storage device. In some embodiments, an appropriate generic model may be identified based on one or more programming parameters for the medical device such as a care area, medication to be administered, use of the medical device (e.g., inpatient, outpatient, acute, and/or non-acute uses) or module used to deliver the medication (e.g., syringe module, large volumetric pump (e.g., delivery of 100 milliliters or more from a single container), etc.). The models may be associated with one or more of the programmed parameters and used to select a model. In some embodiments, the set of models from the model data store device may be narrowed (e.g., filtered) based on one or more values received by the configuration engine, the medical device, the client device, and/or the like. In such instances, the user may be presented with a subset of models that could apply from which a selection of models to actually activate may be received. Similarly, the user may be initially presented with a subset of medications to treat the health condition of the patient, from which the user may select a medication via a display (e.g., the display 54).

In some embodiments, the generic model may receive, as an input, medication information representing a medication selected to be administered to a patient. Additionally and/or alternatively, the input may include an initial health condition of the patient. The generic model may provide, as an output, a generic configuration for the medical device. The generic configuration for the medical device may include one or more dosage parameters, such as a type of medication, a delivery rate (e.g., how frequently to administer the medication, such as a daily rate, a weekly rate, and/or the like), a Volume to Be Infused (VTBI) over a specific time period, a use of the medical device (e.g., inpatient, outpatient, acute, and/or non-acute uses), and/or the like.

At 306, a patient-specific model may be generated, based at least in part on the generic model and the health condition of the patient. The patient-specific model may include one or more predictive models such as a Pk/Pd model and/or a Bayesian-based predictive model. As described herein, the health condition of the patient may be dynamically updated and incorporated into the patient-specific model to more accurately and effectively treat the patient. In some embodiments, the patient-specific model may additionally and/or alternatively be generated based at least in part on one or more patient demographics, such as a patient age, a patient sex, a patient race, a patient ethnicity, a patient height, a patient weight, laboratory results, and a patient gender, whether the health condition of the patient is improving or worsening, medical device data such as one or more medication-specific or diagnostic-specific information, a previous patient-specific model, and/or the like.

At 308, the first information may be provided to the patient-specific model to generate a patient-specific configuration for the medical device. The patient-specific model may then generate the patient-specific configuration.

In some instances, the patient-specific configuration generated by the patient-specific model may be different from the generic configuration generated by the generic model, as the patient-specific model may account for changes in the health condition of the patient. For example, the configuration engine may determine that the health condition of the patient has changed such that the current patient-specific configuration and/or dosage is incorrect. The configuration engine may then generate an alert, cause a change in the dosage of the medication to be administered to the patient, and/or cause a change in operation of the medical device, such as by causing the medical device to stop operation according to the patient-specific configuration and/or by causing the medical device to operate according to an updated patient-specific configuration.

At 310, a configuration recommendation, including the generated patient-specific configuration, may be presented. As described herein with respect to FIG. 2, the display may request confirmation that the generated patient-specific configuration should be transmitted to the medical device. In response to the request, the user, via the display, may confirm the presented configuration recommendation or adjust one or more dosage parameters of the presented configuration recommendation and then confirm the presented configuration recommendation. In some embodiments, the configuration engine may identify the medical device associated with the patient and transmit, to the medical device associated with the patient, a control message including the patient-specific configuration. The transmitted control message may cause the medical device to administer the medication to the patient according to the patient-specific configuration. In some embodiments, the confirmation of the patient-specific configuration is not requested and instead, the patient-specific configuration is automatically transmitted to the medical device.

As noted above, the configuration engine, or another component of the patient monitoring system may determine that the patient-specific configuration is no longer correct, for example, based on a change in the health condition of the patient. The configuration engine may generate an updated patient-specific model. If the updated patient-specific model does not correspond to the existing patient-specific model, the configuration engine may cause the medical device to adjust one or more functions. For example, if one or more parameters provided by the updated patient-specific model does not fall within a predetermined threshold of one or more of the corresponding existing parameters, the configuration engine may cause the medical device to disable power to a motor or other hardware element of the medical device. In some embodiments, the adjustment may include adjusting a user interface or elements presented thereon. For example, a programming user interface may include a start element that, when activated, provides a signal to initiate administration of the medication according to the patient-specific configuration and/or the updated patient-specific configuration. If one or more dosage parameters provided by the patient-specific model are out of range of the one or more updated dosage parameters provided by the updated patient-specific mode, the start element may be deactivated or hidden from the user. The start element may be deactivated or hidden from the user, or the medical device may be disabled until a confirmation is received from the user.

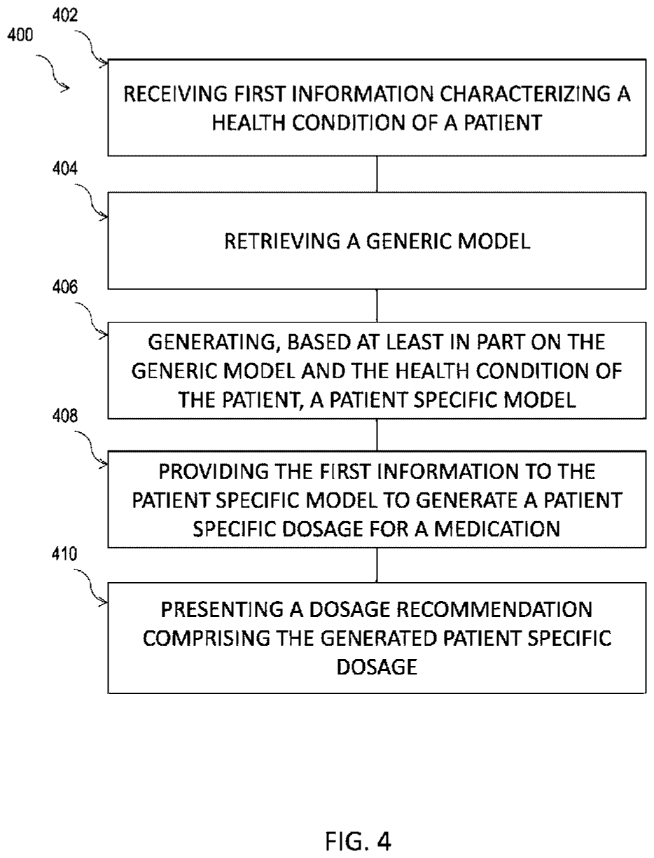

FIG. 4 depicts a flowchart illustrating a process 400 for providing a dosage of a medication, consistent with embodiments of the current subject matter. Referring to FIG. 4, the process 400 may be performed by the patient monitoring system 100, such as by the configuration engine 110.

In some embodiments, the patient monitoring system (e.g., the patient monitoring system 100) is initialized. At 402, the patient monitoring system, such as the configuration engine, may receive first information characterizing a health condition of a patient. The first information may include patient data representing a genetic test result, a genomic test result, a change in organ function of the patient, a severity of illness of the patient, a type of health condition of the patient, an allergy of the patient, a medication prescribed for the patient, a liver function test result, a kidney function test result, and/or a blood cell panel.

At 404, a generic model (e.g., a predictive model including a Pk/Pd model and/or a Bayesian-based predictive model) may be retrieved by the configuration engine from a model data storage device. In some embodiments, an appropriate generic model may be identified based on one or more programming parameters for the medical device such as a care area, medication to be administered, or module used to deliver the medication (e.g., syringe module, large volumetric pump, etc.). The models may be associated with one or more of the programmed parameters and used to select a model. In some embodiments, the set of models from the model data store device may be narrowed (e.g., filtered) based on one or more values received by the configuration engine, the medical device, the client device, and/or the like. In such instances, the user may be presented with a subset of models that could apply from which a selection of models to actually activate may be received. Similarly, the user may be initially presented with a subset of medications to treat the health condition of the patient, from which the user may select a medication via a display (e.g., the display 54).

In some embodiments, the generic model may receive, as an input, medication information representing a medication selected to be administered to a patient. Additionally and/or alternatively, the input may include an initial health condition of the patient. The generic model may provide, as an output, an initial dosage for the medication to be delivered to the patient. The initial dosage may include one or more dosage parameters, such as a type of medication, a delivery rate (e.g., how frequently to administer the medication, such as a daily rate, a weekly rate, and/or the like), a Volume to Be Infused (VTBI) over a specific time period and/or the like.

At 406, a patient-specific model may be generated, based at least in part on the generic model and the health condition of the patient. The patient-specific model may include one or more predictive models such as a Pk/Pd model and/or a Bayesian-based predictive model. As described herein, the health condition of the patient may be dynamically updated and incorporated into the patient-specific model to more accurately and effectively treat the patient. In some embodiments, the patient-specific model may additionally and/or alternatively be generated based at least in part on one or more patient demographics, such as a patient age, a patient sex, a patient race, a patient ethnicity, a patient height, a patient weight, laboratory results, and a patient gender, whether the health condition of the patient is improving or worsening, medical device data such as one or more medication-specific or diagnostic-specific information, a previous patient-specific model, and/or the like.

At 408, the first information may be provided to the patient-specific model to generate a patient-specific dosage for the medication. The patient-specific model may then generate the patient-specific dosage.

In some instances, the patient-specific dosage generated by the patient-specific model may be different from the initial dosage generated by the generic model, as the patient-specific model may account for changes in the health condition of the patient. For example, the configuration engine may determine that the health condition of the patient has changed such that the current dosage is incorrect. The configuration engine may then generate an alert, cause a change in the dosage of the medication to be administered to the patient, cause a change in dosage of the medication to be prepared, cause a change in dosage of the medication to be accessed at a dispensing cabinet, and/or cause a change in operation of a medical device for administering the medication, such as by causing a medical device to stop operation according to the patient-specific dosage, by causing the medical device to operate according to an updated dosage, by permitting only an updated amount of the medication to be accessed at the dispensing cabinet, and/or the like.

At 410, a dosage recommendation, including the generated patient-specific dosage, may be presented. As described herein with respect to FIG. 2, the display may request confirmation that the generated patient-specific dosage should be transmitted to the medical device, dispensing cabinet, and/or pharmacy. In response to the request, the user, via the display, may confirm the presented dosage recommendation or adjust one or more dosage parameters of the presented dosage recommendation and then confirm the presented dosage recommendation. In some embodiments, the configuration engine may identify the medical device, dispensing cabinet, and/or pharmacy associated with the patient and transmit, to the medical device, dispensing cabinet, and/or pharmacy associated with the patient, the dosage recommendation. In some embodiments, the confirmation of the patient-specific dosage is not requested and instead, the patient-specific dosage is automatically transmitted to the medical device, the dispensing cabinet, and/or the pharmacy. Accordingly, the patient monitoring system described herein may provide more accurate medication delivery configurations and/or dosages for a patient to more effectively, efficiently, and quickly treat a patient.

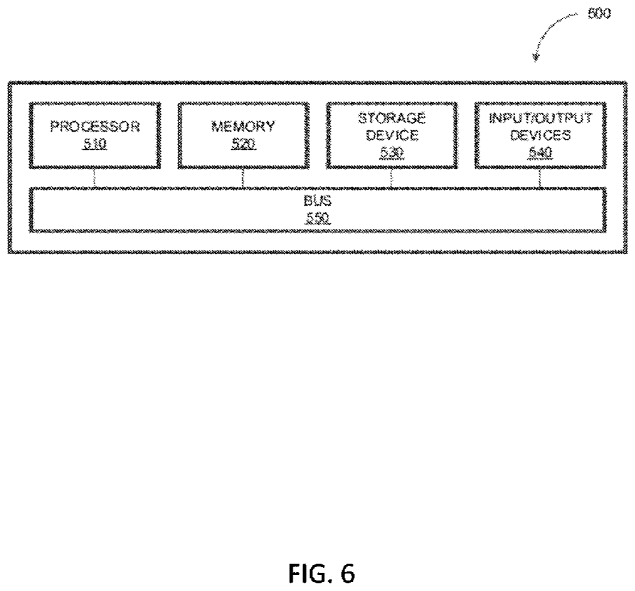

FIG. 6 depicts a block diagram illustrating a computing system 500 consistent with embodiments of the current subject matter. Referring to FIGS. 1A, 1B, and 5, the computing system 500 can be used to implement the patient monitoring system 100 and/or any components therein.

As shown in FIG. 6, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the configuration engine 110. In some example embodiments, the processor 510 can be a single-threaded processor. Alternatively, the processor 510 can be a multi-threaded processor. The processor 510 is capable of processing instructions stored in the memory 520 and/or on the storage device 530 to present graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example embodiments, the input/output device 540 includes a keyboard and/or pointing device. In various embodiments, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

In some example embodiments, the medical device 22, such as a pump 22, may be part of a patient care system 20. FIGS. 7A-7C illustrate example embodiments of the patient care system 20, though other types of patient care systems may be implemented. Referring to FIG. 7A, the patient care system 20 may include the pump 22 as well as additional pumps 24, 26, and 28. Although a large volume pump (LVP) is illustrated, other types of pumps may be implemented, such as a small volume pump (SVP), a TCI pump, a syringe pump, an anesthesia delivery pump, and/or a patient-controlled analgesic (PCA) pump configured to deliver a medication to a patient. The pumps 22, 24, 26, 28 may be configured with one or more patient-specific configurations and/or dosages generated by one or more of the predictive models described herein. In some embodiments, one or more of the predictive models may be stored on the pumps 22, 24, 26, 28. The pump 22 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like, or the pump 22 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like.

As shown in FIG. 7A, each of the pump 22, 24, 26, and 28 may be fluidly connected with an upstream fluid line 30, 32, 34, and 36, respectively. Moreover, each of the four pumps 22, 24, 26, and 28 may also fluidly connected with a downstream fluid line 31, 33, 35, and 37, respectively. The fluid lines can be any type of fluid conduit, such as tubing, through which fluid can flow. At least a portion of one or more of the fluid lines may be constructed with a multi-layered configuration as described herein.

Fluid supplies 38, 40, 42, and 44, which may take various forms but in this case are shown as bottles, are inverted and suspended above the pumps. Fluid supplies may also take the form of bags, syringes, or other types of containers. Both the patient care system 20 and the fluid supplies 38, 40, 42, and 44 may be mounted to a roller stand or intravenous (IV) pole 46.

A separate pump 22, 24, 26, and 28 may be used to infuse each of the fluids of the fluid supplies into the patient. The pumps 22, 24, 26, and 28 may be flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may comprise drugs or nutrients or other fluids.

Typically, medical fluid administration sets have more parts than are shown in FIG. 7A. Many have check valves, drip chambers, valved ports, connectors, and other devices well known to those skilled in the art. These other devices have not been included in the drawings so as to preserve clarity of illustration. In addition, it should be noted that the drawing of FIG. 7A is not to scale and that distances have been compressed for the purpose of clarity. In an actual setting, the distance between the bottles 38, 40, 42, and 44 and the pump modules 22, 24, 26, and 28 could be much greater.

Referring now to FIG. 7B, an enlarged view of the front of the patient care system 20 is shown. The pump 22 may include a front door 50 and a handle 52 that operates to lock the door in a closed position for operation and to unlock and open the door for access to the internal pumping and sensing mechanisms and to load administration sets for the pump. When the door is open, the tube can be connected with the pump, as will be shown in FIG. 7C. When the door is closed, the tube is brought into operating engagement with the pumping mechanism, the upstream and downstream pressure sensors, and the other equipment of the pump. A display 54, such as an LED display, is located in plain view on the door in this embodiment and may be used to visually communicate various information relevant to the pump, such as alert indications (e.g., alarm messages). The display 54 may otherwise be a part of or be coupled to the pump 22. Control keys 56 exist for programming and controlling operations of the pump as desired. The pump 22 also includes audio alarm equipment in the form of a speaker (not shown).

In the embodiment shown, a programming module 60 is attached to the left side of the pump 22. In some embodiments, the programming module 60 forms a part of the pump 22. Other devices or modules, including another pump, may be attached to the right side of the pump 22, as shown in FIG. 7A. In such a system, each attached pump represents a pump channel of the overall patient care system 20. In one embodiment, the programming module is used to provide an interface between the pump 22 and external devices as well as to provide most of the operator interface for the pump 22.

The programming module 60 includes a display 62 for visually communicating various information, such as the operating parameters of the pump 22 and alert indications and alarm messages, which may indicate that the patient-specific configuration and/or dosage should be updated as a result of a change in the health condition of the patient, for example. The programming module 60 may additionally and/or alternatively display the one or more patient parameters and/or the one or more dosage parameters described herein to the display 54 and/or the display 64. The programming module 60 may also include a speaker to provide audible alarms. The programming module or any other module also has various input devices in this embodiment, including control keys 64 and a bar code or other scanner or reader for scanning information from an electronic data tag relating to the infusion, the patient, the care giver, or other. The programming module also has a communications system (not shown) with which it may communicate with external equipment such as a medical facility server or other computer and with a portable processor, such as a handheld portable digital assistant (“PDA), or a laptop-type of computer, or other information device that a care giver may have to transfer information as well as to download drug libraries to a programming module or pump. In some embodiments, the programming module 60 may communicate with the configuration engine 110, include the configuration engine 110, or implement features of the configuration engine 110 described herein.

The communications system may take the form of a radio frequency (“RF”) (radio frequency) system, an optical system such as infrared, a Blue Tooth system, or other wired or wireless system. The bar code scanner and communications system may alternatively be included integrally with the pump 22, such as in cases where a programming module is not used, or in addition to one with the programming module. Further, information input devices need not be hard-wired to medical instruments, information may be transferred through a wireless connection as well.

FIG. 7B includes a second pump 26 connected to the programming module 60. As shown in FIG. 7A, more pump modules may be connected. Additionally, other types of modules may be connected to the pump modules or to the programming module. In such embodiments, the configuration engine 110 may maintain determine, adjust, and/or display values of each of the one or more patient parameters and/or the dosage parameters for each pump (e.g., pump 22 and pump 26).

Turning now to FIG. 7C, the pump 22 is shown in perspective view with the front door 50 open, showing the upstream fluid line 30 and downstream fluid line 31 in operative engagement with the pump 22. The pump 22 directly acts on a tube 66 (also referred to as a pump segment) that connects the upstream fluid line 30 to the downstream fluid line 31 to form a continuous fluid conduit, extending from the respective fluid supply 38 (FIG. 7A) to the patient 48, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 22, such as the type described in co-pending U.S. patent application Ser. No. 13/827,775, which is incorporated by reference herein.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus at no time is there a direct fluid path from the fluid supply to the patient. The operation of peristaltic pumps including four finger pumps is well known to those skilled in the art and no further operational details are provided here.

In this particular embodiment, FIG. 7C further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 7A) and the flow control device, so that the connection of the correct fluid supply with the correct pump may be verified before any fluid is pumped to the patient.

With reference still to FIG. 7C, an upstream pressure sensor 80 may also be included in the pump 22. The upstream pressure sensor is assigned to the flow control device or pumping mechanism 70 and, in this embodiment, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 7A) and the flow control device, so that the connection of the correct fluid supply with the correct pump may be verified before any fluid is pumped to the patient. In an implementation where the source is a syringe, the flow control device 70 may be configured to press a plunger of the syringe to provide the infusion according to the programmed parameters.

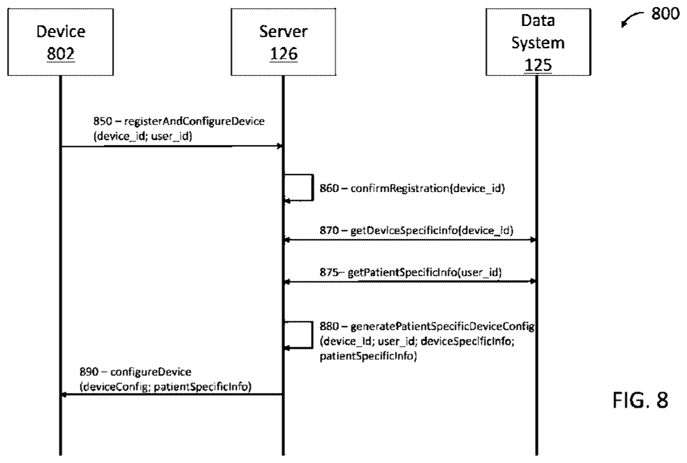

FIG. 8 depicts example messages that may be exchanged to configure a medical device. The messaging system 800 in FIG. 8 includes a device 802 (e.g., the medical device 22), the server 126, and the data system 125. The entities in FIG. 8 are shown exchanging messages directly however, in some implementations, the messages may be mediated or transmitted through one or more intermediary device such as a gateway, proxy server, security scanning device, access point, or other electronic communication device. The device 802 may be an infusion pump (e.g., the medical device 22), a dispensing cabinet (e.g., the dispensing cabinet 104), a diagnostic device (e.g., the diagnostic system 142), a smart sensor such as a Foley catheter, a laboratory system, a clinical decision support module, or other device for use in acute (e.g., hospital) or non-acute (e.g., home care, nursing home, etc.) facilities.

The device 802 may transmit message 850 to the server 126. The message 850 may include an identifier (“device_id”) for the device 802. The device identifier may be a unique identifier that can be used directly or indirectly (e.g., via look up) to determine characteristics of the device 802 such as device type, device model number, software or firmware version for the device 802, configuration of the device 802, and the like. The message 850 may additionally or alternatively include an identifier for a user associated with the device (“user_id”). The user identifier may be used to directly or indirectly (e.g., via look up) determine characteristics of the user such as personal information, personal health information, subscription information, device service level, and the like. The message 850 may include a location identifier. For example, for an acute care device, it may be desirable to use a generic model or configuration that is tailored to a clinic or practice associated with the location identifier.

The server 126 may perform messaging 860 to confirm registration based at least in part on the received device identifier. The confirmation may include confirming the message 850 is authentic. The authenticity may be confirmed using tokens, security keys, two-factor verification, public-private key pair, or the like. The confirmation may include verifying the device identifier is valid (e.g., associated with a device produced). The confirmation may include verifying the device 802 associated with the identifier is functional (e.g., not recalled, defective, out of date, out of operational compliance, not stolen, etc.). The confirmation may be performed directly, in whole or in part, by the server 126. In some implementations, the server 126 may cause a third-party registry system to perform at least part of the confirmation. For example, a manufacturer of the device 802 may provide a service interface for confirming device identifiers. In response to a confirmation request, the manufacturer service may provide a code indicating the status of the device identifier.

The messaging system 800 shown in FIG. 8 assumes the device identifier is valid and confirmed. In cases where the identifier is not valid, the server 126 may transmit a message to the device 802 indicating the same. In some implementations, the message may include a request for additional information for a subsequent attempt at registering and configuring the device 802 (e.g., alternate device identifier).

Having confirmed the registration, the server 127 may exchange one or more messages 870, with the data system to acquire device specific information. For example, the data system 125 may be a configuration model associated with the device. The model received from the data system 125 may be a generic model for generating a generic configuration for the device. The message 870 may include a location identifier such as the location identifier included in message 850. For example, for an acute care device, it may be desirable to use a generic model or configuration that is tailored to a clinic or practice associated with the location identifier.

The server 126 may exchange one or more messages 875 with the data system 125 to acquire patient specific information. The messages 875 may include the user identifier received in message 875. FIG. 8 shows message 875 between the server 126 and one data system. However, in some implementations, the server 126 may communicate with multiple data systems to collect the information needed to generate a configuration for the device 802. For example, the server 126 may first query an electronic health records system for a hospital to obtain an identifier for a laboratory order for the user. The server 126 may then transmit a message to a laboratory information system including the identifier to obtain results for the user.

Based on at least a portion of the patient specific information and the device specific information, the server 126 may generate a patient specific device configuration via messaging 880. The generation of the configuration may be performed in whole or in part using one or more of the methods described such as those in FIG. 3, 4, or 5.

The server 126 may then cause the device 802 to be configured according to the configuration generated via messaging 880. This may include transmitting the patient specific model to the device 802. This may include generating specific configuration parameters using the patient specific model and providing the parameters to the device 802. In some implementations, the configuration may include a timestamp indicating when the configuration was generated. In some implementations, the configuration may include an expiration date indicating the time or amount of time the configuration is valid for. For example, the configuration may be generated based on a physiological conditional of the user that may change. In such instances, the configuration may need to be evaluated periodically to ensure the patient specific values keep pace with any changes to the user's condition.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and one or more hardware buttons, a keyboard and/or a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices, hardware buttons, and associated interpretation software, and the like.

Although the disclosure, including the figures, described herein may describe and/or exemplify different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of configuring a medical device:
receiving first information characterizing a health condition of a patient;
retrieving, from a model data storage device, a generic model, wherein the generic model receives, as an input, medication information, and wherein the generic model provides, as an output, a generic configuration for the medical device;
generating, based at least in part on the generic model and the health condition of the patient, a patient-specific model;
providing the first information to the patient-specific model to generate a patient-specific configuration for the medical device; and
presenting a configuration recommendation, the configuration recommendation comprising the generated patient-specific configuration.

2. The method of claim 1, further comprising:
identifying the medical device associated with the patient; and
transmitting, to the medical device associated with the patient, a control message including the patient-specific configuration.

3. The method of any one of claims 1 to 2, wherein the patient-specific configuration is different from the generic configuration.

4. The method of any one of claims 1 to 3, further comprising: requesting confirmation that the generated patient-specific configuration should be transmitted to the medical device.

5. The method of claim 4, further comprising: receiving, in response to the requested confirmation, the confirmation.

6. The method of claim 4, further comprising: receiving, in response to the requested confirmation, an adjustment of the patient-specific configuration.

7. The method of any one of claims 1 to 6, further comprising: generating the patient-specific configuration for the medical device.

8. The method of any one of claims 1 to 7, further comprising:
receiving second information characterizing a second health condition of the patient at a second time that is after a first time of the first health condition;
generating, based at least in part on the patient-specific model and the second information, an updated patient-specific model; and
providing the second information to the updated patient-specific model to generate an updated patient-specific configuration for the medical device.

9. The method of claim 8, further comprising: requesting confirmation that the updated patient-specific configuration should be provided to the medical device.

10. The method of claim 8, further comprising: generating an alert indicating that an updated patient-specific configuration has been generated.

11. The method of claim 8, further comprising: determining that the second health condition is different from the first health condition.

12. The method of claim 11, further comprising: generating an alert indicating that the patient-specific configuration should be updated.

13. The method of claim 11 or claim 12, further comprising: causing a change in operation of the medical device.

14. The method of claim 11 or claim 12, further comprising: causing the medical device to stop operation according to the patient-specific configuration.

15. The method of claim 11 or claim 12, further comprising: causing the medical device to operate according to the updated patient-specific configuration.

16. The method of any one of claims 1 to 15, wherein the first information comprises patient data, the patient data representing one or more of a genetic test result, a genomic test result, a change in organ function of the patient, a severity of illness of the patient, a type of health condition of the patient, an allergy of the patient, a medication prescribed for the patient, a liver function test result, a kidney function test result, a molecular test result, a drug level test result, and a blood cell panel.

17. The method of any one of claims 1 to 16, wherein the patient-specific model is further generated based at least in part on one or more patient demographics, the one or more patient demographics comprising a patient age, a patient sex, a patient race, a patient ethnicity, a patient height, a patient weight, and a patient gender.

18. The method of any one of claims 1 to 17, wherein the patient-specific model is further generated based at least in part on whether the health condition of the patient is improving or worsening.

19. The method of any one of claims 1 to 18, wherein the patient-specific model is further generated based at least in part on medical device data, the medical device data comprising one or more medication-specific or diagnostic-specific information.

20. The method of any one of claims 1 to 19, wherein the generic model comprises one or more of a pharmacokinetic model, a pharmacodynamics model, and a Bayesian-based model.

21. The method of any one of claims 1 to 20, further comprising: causing, based on the patient-specific configuration information, administration of a medication to the patient.

22. A method comprising:
receiving first information characterizing a health condition of a patient;
retrieving, from a model data storage device, a generic model, wherein the generic model receives, as an input, medication information for a medication, and wherein the generic model provides, as an output, an initial dosage for the medication to be delivered to the patient;
generating a patient-specific model based at least in part on the generic model and the health condition of the patient;
providing the first information to the patient-specific model to generate a patient-specific dosage for the medication; and
presenting a dosage recommendation for the medication, the dosage recommendation comprising the patient-specific dosage.

23. The method of claim 22, further comprising: determining that the patient-specific dosage is different from the initial dosage.

24. The method of claim 23, further comprising: generating, based on the determination, an alert indicating that the patient-specific dosage is different from the initial dosage.

25. The method of claim 24, further comprising: requesting, after determining that the patient-specific dosage is different from the initial dosage, confirmation of the patient-specific dosage.

26. The method of claim 25, further comprising: transmitting, based on the confirmation of the patient-specific dosage, the patient-specific dosage to a medical device associated with the patient.

27. The method of claim 25, further comprising: transmitting, based on the confirmation of the patient-specific dosage, the patient-specific dosage to a dispensing cabinet, the dispensing cabinet configured to allow the patient-specific dosage to be dispensed.

28. The method of claim 25, further comprising: transmitting, based on the confirmation of the patient-specific dosage, the patient-specific dosage to a pharmacy information system.

29. The method of claim 25, wherein the patient-specific dosage comprises one or more of a medication type, an amount of the medication, a time frequency for delivering the medication, and an amount of time for delivering the medication.

30. A system, comprising:
at least one data processor; and
at least one memory storing instructions which, when executed by the at least one data processor, result in operations comprising:
receiving first information characterizing a health condition of a patient;
retrieving, from a model data storage device, a generic model, wherein the generic model receives, as an input, medication information, and wherein the generic model provides, as an output, a generic configuration for the medical device;
generating, based at least in part on the generic model and the health condition of the patient, a patient-specific model;
providing the first information to the patient-specific model to generate a patient-specific configuration for the medical device; and
presenting a configuration recommendation, the configuration recommendation comprising the generated patient-specific configuration.

31. The system of claim 30, wherein the operations further comprise:
identifying the medical device associated with the patient; and
transmitting, to the medical device associated with the patient, a control message including the patient-specific configuration.

32. The system of any one of claims 30 to 31, wherein the patient-specific configuration is different from the generic configuration.

33. The system of any one of claims 30 to 32, wherein the operations further comprise: requesting confirmation that the generated patient-specific configuration should be transmitted to the medical device.

34. The system of claim 33, wherein the operations further comprise: receiving, in response to the requested confirmation, the confirmation.

35. The system of claim 33, wherein the operations further comprise: receiving, in response to the requested confirmation, an adjustment of the patient-specific configuration.

36. The system of any one of claims 30 to 35, wherein the operations further comprise: generating the patient-specific configuration for the medical device.

37. The system of any one of claims 30 to 36, wherein the operations further comprise:
receiving second information characterizing a second health condition of the patient at a second time that is after a first time of the first health condition;
generating, based at least in part on the patient-specific model and the second information, an updated patient-specific model; and
providing the second information to the updated patient-specific model to generate an updated patient-specific configuration for the medical device.

38. The system of claim 37, wherein the operations further comprise: requesting confirmation that the updated patient-specific configuration should be provided to the medical device.

39. The system of claim 37, wherein the operations further comprise: generating an alert indicating that an updated patient-specific configuration has been generated.

40. The system of claim 37, wherein the operations further comprise: determining that the second health condition is different from the first health condition.

41. The system of claim 40, wherein the operations further comprise: generating an alert indicating that the patient-specific configuration should be updated.

42. The system of claim 40 or claim 41, wherein the operations further comprise: causing a change in operation of the medical device.

43. The system of claim 40 or claim 41, wherein the operations further comprise: causing the medical device to stop operation according to the patient-specific configuration.

44. The system of claim 40 or claim 41, wherein the operations further comprise: causing the medical device to operate according to the updated patient-specific configuration.

45. The system of any one of claims 30 to 44, wherein the first information comprises patient data, the patient data representing one or more of a genetic test result, a genomic test result, a change in organ function of the patient, a severity of illness of the patient, a type of health condition of the patient, an allergy of the patient, a medication prescribed for the patient, a liver function test result, a kidney function test result, a molecular test result, a drug level test result, and a blood cell panel.

46. The system of any one of claims 30 to 45, wherein the patient-specific model is further generated based at least in part on one or more patient demographics, the one or more patient demographics comprising a patient age, a patient sex, a patient race, a patient ethnicity, a patient height, a patient weight, and a patient gender.

47. The system of any one of claims 30 to 46, wherein the patient-specific model is further generated based at least in part on whether the health condition of the patient is improving or worsening.

48. The system of any one of claims 30 to 47, wherein the patient-specific model is further generated based at least in part on medical device data, the medical device data comprising one or more medication-specific or diagnostic-specific information.

49. The system of any one of claims 30 to 48, wherein the generic model comprises one or more of a pharmacokinetic model, a pharmacodynamics model, and a Bayesian-based model.

50. The system of any one of claims 30 to 49, wherein the operations further comprise: causing, based on the patient-specific configuration information, administration of a medication to the patient,

51. A system, comprising:
at least one data processor; and
at least one memory storing instructions which, when executed by the at least one data processor, result in operations comprising:
receiving first information characterizing a health condition of a patient;
retrieving, from a model data storage device, a generic model, wherein the generic model receives, as an input, medication information for a medication, and wherein the generic model provides, as an output, an initial dosage for the medication to be delivered to the patient;
generating a patient-specific model based at least in part on the generic model and the health condition of the patient;
providing the first information to the patient-specific model to generate a patient-specific dosage for the medication; and
presenting a dosage recommendation for the medication, the dosage recommendation comprising the patient-specific dosage.

52. The system of claim 51, wherein the operations further comprise: determining that the patient-specific dosage is different from the initial dosage.

53. The system of claim 52, wherein the operations further comprise: generating, based on the determination, an alert indicating that the patient-specific dosage is different from the initial dosage.

54. The system of claim 53, wherein the operations further comprise: requesting, after determining that the patient-specific dosage is different from the initial dosage, confirmation of the patient-specific dosage.

55. The system of claim 54, wherein the operations further comprise: transmitting, based on the confirmation of the patient-specific dosage, the patient-specific dosage to a medical device associated with the patient.

56. The system of claim 54, wherein the operations further comprise: transmitting, based on the confirmation of the patient-specific dosage, the patient-specific dosage to a dispensing cabinet, the dispensing cabinet configured to allow the patient-specific dosage to be dispensed.

57. The system of claim 54, wherein the operations further comprise: transmitting, based on the confirmation of the patient-specific dosage, the patient-specific dosage to a pharmacy information system.

58. The system of claim 54, wherein the patient-specific dosage comprises one or more of a medication type, an amount of the medication, a time frequency for delivering the medication, and an amount of time for delivering the medication.

59. A non-transitory computer-readable storage medium including program code, which when executed by at least one data processor, cause operations comprising:
receiving first information characterizing a health condition of a patient;
retrieving, from a model data storage device, a generic model, wherein the generic model receives, as an input, medication information, and wherein the generic model provides, as an output, a generic configuration for the medical device;
generating, based at least in part on the generic model and the health condition of the patient, a patient-specific model;
providing the first information to the patient-specific model to generate a patient-specific configuration for the medical device; and
presenting a configuration recommendation, the configuration recommendation comprising the generated patient-specific configuration.

60. A non-transitory computer-readable storage medium including program code, which when executed by at least one data processor, cause operations comprising:
receiving first information characterizing a health condition of a patient;
retrieving, from a model data storage device, a generic model, wherein the generic model receives, as an input, medication information for a medication, and wherein the generic model provides, as an output, an initial dosage for the medication to be delivered to the patient;
generating a patient-specific model based at least in part on the generic model and the health condition of the patient;
providing the first information to the patient-specific model to generate a patient-specific dosage for the medication; and
presenting a dosage recommendation for the medication, the dosage recommendation comprising the patient-specific dosage.

61. An apparatus, comprising:
means for receiving first information characterizing a health condition of a patient;
means for retrieving, from a model data storage device, a generic model, wherein the generic model receives, as an input, medication information, and wherein the generic model provides, as an output, a generic configuration for the medical device;
means for generating, based at least in part on the generic model and the health condition of the patient, a patient-specific model;
means for providing the first information to the patient-specific model to generate a patient-specific configuration for the medical device; and
means for presenting a configuration recommendation, the configuration recommendation comprising the generated patient-specific configuration.

62. The apparatus of claim 61, comprising:
means for performing any of the functions recited in any of claims 2 to 21.

63. An apparatus, comprising:
means for receiving first information characterizing a health condition of a patient;
means for retrieving, from a model data storage device, a generic model, wherein the generic model receives, as an input, medication information for a medication, and wherein the generic model provides, as an output, an initial dosage for the medication to be delivered to the patient;
means for generating a patient-specific model based at least in part on the generic model and the health condition of the patient;
means for providing the first information to the patient-specific model to generate a patient-specific dosage for the medication; and
means for presenting a dosage recommendation for the medication, the dosage recommendation comprising the patient-specific dosage.

64. The apparatus of claim 63, comprising:
means for performing any of the functions recited in any of claims 23 to 29.

65. A system, comprising:
at least one data processor; and
at least one memory storing instructions which, when executed by the at least one data processor, result in operations comprising:
receiving information characterizing a health condition of one or more patients at a medical facility;
generating, based on the information, a metric for the medical facility;
determining whether the metric corresponds to a safety threshold; and
scheduling, based on the determination, a next review for the facility.

66. The system of claim 65, wherein the operations further comprise:
determining that the metric does not correspond to the safety threshold;
identifying a facility configuration for updating; and
initiating an update of the facility configuration.

67. The system of claim 65, wherein the operations further comprise:
determining that the metric corresponds to the safety threshold.
